SEQUENCE LISTING (11 sequences)

SEQ ID NO: 1 - DNA, 30 bp, Artificial Sequence
  Description of Artificial Sequence Synthetic primer
aggaattcgaatggctgagcaggagcccac

SEQ ID NO: 2 - DNA, 30 bp, Artificial Sequence
  Description of Artificial Sequence Synthetic primer
cgggatcctcatcagtccttccagtccttc

SEQ ID NO: 3 - DNA, 27 bp, Artificial Sequence
  Description of Artificial Sequence Synthetic primer
aggaattcgaaacgtcgtggtgactgg

SEQ ID NO: 4 - DNA, 34 bp, Artificial Sequence
  Description of Artificial Sequence Synthetic primer
cgggatcctcatcagtcaatcttgacgcctttcc

SEQ ID NO: 5 - DNA, 31 bp, Artificial Sequence
  Description of Artificial Sequence Synthetic primer
aggaattcgaaagactgactacatggtaggc

SEQ ID NO: 6 - DNA, 22 bp, Artificial Sequence
  Description of Artificial Sequence Synthetic primer
ttcttgaaggcctccaggtcgc

SEQ ID NO: 7 - DNA, 21 bp, Artificial Sequence
  Description of Artificial Sequence Synthetic primer
acctggaggccttcaagaagc

SEQ ID NO: 8 - DNA, 20 bp, Artificial Sequence
  Description of Artificial Sequence Synthetic primer
gcgggacttgatggcgtagc

SEQ ID NO: 9 - DNA, 21 bp, Artificial Sequence
  Description of Artificial Sequence Synthetic primer
agctacgccatcaagtcccgc

SEQ ID NO: 10 - DNA, 22 bp, Artificial Sequence
  Description of Artificial Sequence Synthetic primer
tccttcttgatggcgagattcc

SEQ ID NO: 11 - DNA, 21 bp, Artificial Sequence
  Description of Artificial Sequence Synthetic primer
aatctcgccatcaagaaggac

(19) United States
(12) Patent Application Publication
(10) Pub. No.: US 2008/0160552 A1
(43) Pub. Date: Jul. 3, 2008

(54) Methods and Compounds for Treating Inflammation

(76) Inventors: Dolly Mehta, Elmhurst, IL (US); Nebojsa Knezevic, Chicago, IL (US); Asrar Malik, Hinsdale, IL (US)

Correspondence Address: LARRY S. MILLSTEIN;HOLLAND & KNIGHT LLP, 1600 TYSONS BOULEVARD, SUITE 700, MCLEAN, VA, 22102, US

(21) Appl. No.: 11/954,933

(22) Filed: Dec. 12, 2007

Related U.S. Application Data
(60) Provisional application No. 60/874,584, filed on Dec. 13, 2006.

(51) Int. Cl. C12Q 1/48; A61K 31/47

(52) U.S. Cl. 435/15; 514/789; 514/310

(57) ABSTRACT

The invention relates in embodiments to methods for identifying compounds that inhibit phosphorylation of GD-1, to compositions comprising such inhibitors, including pharmaceutical compositions, and to the use of such inhibitors and compositions to treat diseases, such as inflammatory diseases.

### REFERENCE TO RELATED APPLICATION

This application claims full benefit of priority of U.S. Provisional Application Ser. No. 60/874,584 filed on 13 Dec. 2006 which is herein incorporated by reference in its entirety

### GOVERNMENT RIGHTS

This work was supported by National Institutes of Health Grants HL 45638 and 71794. The U.S. Government has certain rights in this invention.

### TECHNICAL FIELD

The present invention relates to compounds and methods useful in selectively modulating RhoA and GDI-1 activity and RhoA-dependent increases and decreases in endothelial permeability.

### BACKGROUND OF THE INVENTION

The monomeric RhoGTPase RhoA plays a critical role in regulating cell migration, contraction, growth, and apoptosis (29, 36). In endothelial cells, RhoA activity is required to increase vascular permeability (22). Guanine nucleotide dissociation inhibitors (GDIs) bind with the GDP form of RhoGTPases and prevent their dissociation from the complex. Thus, Rho-GDI interaction serves as a primary mechanism for limiting RhoGTPase activation (5, 8, 26). GDIs also stimulate the release of RhoGTPases from cell membranes, thereby inactivating the RhoGTPase cycle (5, 8, 26).

Rho-GDI-1 is an ubiquitously expressed member of the Rho-GDI family (5, 8, 26). Rho-GDI-1 is composed of a flexible 69 amino acid long N-terminus and a folded 135 amino acid long C-terminus (5, 8, 26). The C-terminus domain contains a hydrophobic pocket that binds to the isoprenyl group of RhoGTPases and brings GTPases in close proximity of N-terminus (Gosser, 1997 #79; Faure, 2001 #90; Hoffman, 2000 #80). This conformation change appears to be required for formation of a stable GDI-RhoGTPase complex and restricting the activation of RhoGTPases (8, 11, 20, 21). While structural analysis has helped to elucidate the basis of GDI-1 inhibition of RhoGTPase activity, the mechanisms by which G-protein coupled receptors destabilize the GDI-RhoGTPase complex, thereby resulting Rho activation, have not been addressed.

Studies implicate GDI-1 phosphorylation, protein-protein interactions, and local increase in phospholipid content in regulating the inhibitory activity of GDI-1 on RhoGTPases (5, 8, 26). The N-terminal domain of radixin, a member of ezrin-radixin-moesin (ERM) family of proteins, binds RhoGDI-1 (13). Transduction of N-terminus radixin in fibroblasts resulted in RhoA-dependent increase in actin stress fiber formation (28), suggesting a role of radixin in regulating GDI-1 interaction with RhoGTPases. Phosphorylation of GDI-1 at Ser101/Ser174 by p21-activated kinase (PAK), an effector of Cdc42, was shown to induce dissociation of Rac1 from Rac1-GDI-1 complex (6), implicating PAK phosphorylation of GDI-1 is an another mechanism of GDI-1 activation. We showed that thrombin by binding to the receptor PAR-1 induced PKCα-mediated GDI-1 phosphorylation resulting in RhoA activation (23). In the present study, based on analysis of RhoGDI-1, we surmised that a phosphorylation switch was required to release RhoA from GDI-1 resulting in RhoA activation. We show herein that Ser96 at the C-terminus is responsible for PKCα-dependent phosphorylation of GDI-1 and thereby selectively induces RhoA activation. The results demonstrate that modulation of RhoA activation by interfering with Ser96 phosphorylation of Rho-GDI-1 prevents the downstream effect of RhoA activation in increasing endothelial permeability.

### SUMMARY OF THE INVENTION

One aspect of the invention provides a method for treating a subject that has or is at risk of developing an inflammatory disease. More specifically, the invention relates to the use of chemical compounds and pharmaceutical compositions that inhibit the enzymatic activity of GDI-1 for the treatment and/or prevention of inflammatory diseases induced by endothelial dysfunction, such as acute lung injury and its more severe form acute respiratory distress syndrome (ARDS), retinopathy, inflammatory bowel disease, arthritis, atherosclerosis, asthma, allergy, inflammatory kidney disease, circulatory shock, multiple sclerosis, chronic obstructive pulmonary disease, skin inflammation, periodontal disease, psoriasis and T cell-mediated diseases of immunity. Examples of T cell -mediated diseases of immunity include allergic encephalomyelitis, allergic neuritis, transplant allograft rejection, graft versus host disease, myocarditis, thyroiditis, nephritis, systemic lupus erthematosus, and insulin-dependent diabetes mellitus.

The invention also provides methods for treating inflammation. “Treatment”, “treating” or “treated” as used herein, means preventing, reducing or eliminating at least one symptom or complication of inflammation. These methods include administering to a subject in need thereof a composition comprising an agent that modulates the activity of GDI-1. In one embodiment, the subject is a human. In one embodiment, this comprises administering a therapeutic amount of an agent that decreases the activity of GDI-1, such as the protein activity or the protein or RNA level of GDI-1.

A “therapeutic amount” represents an amount of an agent that is capable of inhibiting or decreasing the activity or expression of GDI-1. The clinical response includes an improvement in the condition treated or in the prevention of the condition. The particular dose of the agent administered according to this invention will, of course, be determined by the particular circumstances surrounding the case, including the agent administered, the particular inflammatory disease being treated and similar conditions. In some embodiments, the agent interacts with the GDI-1 protein. In one embodiment, the agent is an inhibitor of GDI-1. In other embodiments, the agent interacts and inhibits the a transcription factor responsible for GDI-1 transcription. In still other embodiments, the agent binds to or interacts with (such as by chemically modifying) an inhibitor or activator of GDI's activity or expression. By way of nonlimiting example, an agent may bind to and inhibit an activator of GDI-1 or an agent may bind to and activate an inhibitor of GDI-1 activity. In another embodiment, a “therapeutic amount” represents an amount of an agent that is capable of increasing phosphorylation of GDI-1. For example, an agent may bind to GDI-1 and induce a conformation change in which the GDI-1 protein mimics a phosphorylated form of the protein. Alternatively, an agent may increase phosphorylation of the GDI-1 protein.

In another aspect, the invention provides a pharmaceutical composition comprising an agent that modulates GDI-1 activity and a pharmaceutically acceptable carrier. The pharmaceutical composition can be used for treating inflammation and/or inflammatory diseases. Nonlimiting examples of inflammation that can be treated by this method include of inflammatory bowel disease, arthritis, atherosclerosis, asthma, allergy, inflammatory kidney disease, circulatory shock, multiple sclerosis, chronic obstructive pulmonary disease, skin inflammation, periodontal disease, psoriasis and T cell-mediated diseases of immunity. The agent may be any of the agents described herein or discovered by methods described herein. In some embodiments, the agent inhibits the activity of RhoA protein. In still other embodiments, the agent activates RhoA.

The agent may be administered by a wide variety of routes. Exemplary routes of administration include oral, parenteral, transdermal, and pulmonary administration. For example, the agents may be administered intranasally, intramuscularly, subcutaneously, intraperitonealy, intravaginally and any combination thereof. For pulmonary administration, nebulizers, inhalers or aerosol dispensers may be used to deliver the therapeutic agent in an appropriate formulation (e.g., with an aerolizing agent). In addition, the agent may be administered alone or in combination with other agents, known drugs, or treatment methods. In combination, agents may be administered simultaneously or each agent may be administered at different times. When combined with one or more known anti-inflammatory drugs, agents and drugs may be administered simultaneously or the agent can be administered before or after the drug(s).

In one embodiment, the agents are administered in a pharmaceutically acceptable carrier. Any suitable carrier known in the art may be used. Carriers that efficiently solubilize the agents are preferred. Carriers include, but are not limited to a solid, liquid or a mixture of a solid and a liquid. The carriers may take the form of capsules, tablets, pills, powders, lozenges, suspensions, emulsions or syrups. The carriers may include substances that act as flavoring agents, lubricants, solubilizers, suspending agents, binders, stabilizers, tablet disintegrating agents and encapsulating materials.

Tablets for systemic oral administration may include excipients, as known in the art, such as calcium carbonate, sodium carbonate, sugars (e.g., lactose, sucrose, mannitol, sorbitol), celluloses (e.g., methyl cellulose, sodium carboxymethyl cellulose), gums (e.g., arabic, tragacanth), together with disintegrating agents, such as maize, starch or alginic acid, binding agents, such as gelatin, collagen or acacia and lubricating agents, such as magnesium stearate, stearic acid or talc.

In powders, the carrier is a finely divided solid, which is mixed with an effective amount of a finely divided agent.

In solutions, suspensions, emulsions or syrups, an effective amount of the agent is dissolved or suspended in a carrier such as sterile water or an organic solvent, such as aqueous propylene glycol. Other compositions can be made by dispersing the inhibitor in an aqueous starch or sodium carboxymethyl cellulose solution or a suitable oil known to the art.

The agents are administered in a therapeutic amount. Such an amount is effective in treating an inflammatory disease. This amount may vary, depending on the activity of the agent utilized, the location and stage of the inflammatory disease, and the health of the patient. The term “therapeutically effective amount” is used to denote treatments at dosages effective to achieve the therapeutic result sought. Furthermore, a skilled practitioner will appreciate that the therapeutically effective amount of the agent may be lowered or increased by fine-tuning and/or by administering more than one agent, or by administering an agent with another compound. Therapeutically effective amounts may be easily determined, for example, empirically by starting at relatively low amounts and by step-wise increments with concurrent evaluation of beneficial effect. (e.g., reduction in symptoms).

When one or more agents or anti-inflammatory compounds are combined with a carrier, they may be present in an amount of about 1 weight percent to about 99 weight percent, the remainder being composed of the pharmaceutically acceptable carrier.

### FIGURES AND DRAWINGS

FIG. 1. GDI-1 expression prevents SRE activation and actin stress fiber formation. A, effect of GDI-1 on SRE activity induced by thrombin. HPAE cells were co-transfected with SRE-luciferase plasmid and GFP or GFP-tagged full-length (FL) GDI-1. Cells were then stimulated with thrombin for 5 hr prior to SRE activity measurement using dual reporter assay kit. SRE-luciferase activity is expressed as the ratio of firefly and Renilla luciferase activity. Data represent the mean±SEM from four experiments performed in triplicate. B, effect of GDI-1 on SRE activity induced by constitutively active heterotrimeric G-proteins. HPAEC were co-transfected with constitutively active Gα_{q}, Gα₁₂ or Gα₁₃ without or with FL-GDI-1 and SRE reporter activity was determined. SRE-luc activity is expressed as the ratio of firefly and Renilla luciferase activity. Data represent mean±SEM from three experiments performed in triplicate. C, effects of GDI-1 on thrombin-induced actin stress fiber formation. HPAE cells transfected with GFP or GFP-GDI-1 were stimulated with 50 nM thrombin for 5 min after which cells were fixed and stained with phalloidin to determine actin stress fiber formation. Results are representative of at least three experiments. D, effect of GDI-1 on SRE activation induced by constitutively active RhoA. HPAEC were co-transfected with constitutively active RhoA (V14RhoA) without or with FL-GDI-1, and SRE reporter activity was determined. SRE-luc activity is expressed as the ratio of firefly and Renilla luciferase activity. Data represent the mean±SEM from three experiments performed in triplicate. *, values different from control cells (p<0.05); +, presence; −, absence.

FIG. 2. Effects of FL-GDI-1 or GDI-1 C-terminus on thrombin-induced RhoA activity. A, Top, autoradiogram showing PKCα phosphorylates C-terminus but not N-terminus of GDI-1. Purified FL-GDI-1, or GDI-1 lacking C terminus (aa 69-204) or N-terminus (aa 1-68) proteins were incubated with PKCα in vitro and phosphorylation was determined as described in Methods. A, Bottom, coomasie blue stained gel of GDI-1 proteins. B-C, RhoA activity in response to thrombin stimulation in HPAE cells transduced with GFP-tagged FL- or C-terminus GDI-1. RhoA activity was determined after 2 min of thrombin stimulation. RhoA activation is measured by the increase in amount of GTP-bound RhoA (top) compared to total amount of RhoA in whole cell lysates (middle). Bottom panel shows Western blot of HPAE cells with anti-GFP Ab showing the expression of GDI-1 mutants. C, Plot shows mean±SEM for thrombin-induced increase in RhoA activity from multiple experiments calculated as fold increase over basal value under various experimental conditions (n=3). * indicates increase in RhoA activity compared to unstimulated monolayer (p<0.05).

FIG. 3. PKCα-induced phosphorylation of C-terminus regulates thrombin-induced SRE generation. A, Autoradiogram showing PKCα-induced phosphorylation of C1 and C2 GDI-1 mutants in vitro. COS-7 cells were transduced with the indicated mutants and after 48 hrs lysates were immunoprecipitated with anti-GFP Ab followed by addition of protein A/G plus beads. The immunocomplexes were used for in vitro phosphorylation by PKCα as described in Methods. Compared to C2 domain, PKCα markedly phosphorylated C1 domain of GDI-1. Bottom panel shows the Western blot using anti-GFP Ab indicating equal protein loading. Data are representative of three independent experiments. B, SRE activity in HPAE cells transducing C1- or C2-GDI-1 mutants. HPAE cells were co-transfected with SRE-luciferase plasmid and GFP or indicated GFP-tagged C1- or C2- GDI-1 mutants. Cells were then stimulated with thrombin for 5 hr prior to SRE activity measurement using the dual reporter assay kit. SRE-luciferase activity is expressed as the ratio of firefly and Renilla luciferase activity in response to thrombin from unstimulated cells. Data represent mean±SEM from four experiments performed in triplicate. * indicates increased SRE activity compared to unstimulated monolayer (p<0.05). C, Transduction of kinase-dead PKCα mutant in HPAE cells transducing C1- or C2-GDI-1 mutant rescues the inhibitory activity of C-terminus on RhoA. HPAE cells co-transfected with GFP-tagged C1- or C2-GDI-1 mutant without or with kinase-defective PKCα mutant were stimulated with thrombin for 5 hr prior to SRE activity measurement using the dual reporter assay kit. SRE-luciferase activity is expressed as the ratio of firefly and Renilla luciferase activity quantified as fold increase over unstimulated cells. Data represent mean±SEM from three experiments performed in triplicate. * indicates increased SRE activity compared to unstimulated monolayer (p<0.05).

FIG. 4. Effects of non-phosphorylatable GDI-1 mutants on SRE activity induced by thrombin. A, Mutation of Ser 96, Ser176, and Thr197 to alanine markedly reduced the phosphorylation of C1 or C2 domain by PKCα in vitro. COS-7 cells transduced with the indicated mutants were immunoprecipitated with anti-GFP Ab and complexed with protein A/G plus beads. The immunocomplexes were used for in vitro phosphorylation by PKCα as described in Methods. Bottom panel shows the Western blot using anti-GFP Ab indicating equal protein loading. Data are representative of three independent experiments. B, HPAE cells co-transfected with indicated GFP-tagged mutants were stimulated with thrombin for 5 hr prior to SRE activity measurement using dual reporter assay kit. SRE-luciferase activity is expressed as the ratio of firefly and Renilla luciferase activity quantified as % increase in SRE activity over that in unstimulated cells. Data represent mean±SEM from three experiments performed in triplicate. * indicates increased SRE activity compared to unstimulated monolayer (p<0.05).

FIG. 5. C1-S96A-GDI mutant inhibits RhoA activity. A, HPAE cells transducing C1- or S96A-C1-GDI-1 mutant were stimulated with 50 nM of thrombin for 2 min to determine RhoA activity using GST-bound rhotekin fusion proteins. RhoA activation is measured by the increased amount of GTP-bound RhoA (A, top) compared to GFP expression (A, bottom). B, plot shows mean±SEM for thrombin-induced increase in RhoA activity from multiple experiments calculated as fold increase over basal value under various experimental conditions (n=3). * indicates increase in RhoA activity compared to unstimulated monolayer (p<0.05). C, HPAE cells transducing C1 or S96A-C1 mutant were stimulated with thrombin for 2 min and lysates were immunoprecipitated with anti-GFP Ab followed by Western blotting with anti-RhoA or anti-GFP Abs. Data represent results from at least two experiments.

FIG. 6. C1-S96A-GDI mutant fails to inhibit Rac1 or Cdc42 activity. A, HPAE cells transducing C1- or S96A-C1-GDI-1 mutants were stimulated with 50 nM of thrombin for indicated times to determine Rac1 (A-B) or Cdc42 activity (C-D) using GST-bound PBD fusion proteins. Rac1 or Cdc42 activation is measured by the increased amount of GTP-bound Rac1 (A, top) compared to GFP expression (A, bottom). B, and D, plot shows mean±SEM for thrombin-induced changes in Rac1 or Cdc42 activity from multiple experiments calculated as fold increase over basal value under various experimental conditions (n=3). * indicates increase in GTPase activity compared to unstimulated monolayer (p<0.05).

FIG. 7. Effects of C1-S96A mutant on MLC phosphorylation, actin stress fiber formation, and loss of endothelial barrier function in response to thrombin. A-B, MLC phosphorylation in response to thrombin in cells transfected with C1 or C1-S96A-GDI-1 mutant. Cells were stimulated with thrombin for indicated time and lysates were Western blotted with anti-phospho-MLC (top) or anti-GFP Abs to determine MLC phosphorylation. B, plot shows mean increase in MLC phosphorylation calculated as fold increase over basal value under various experimental conditions (n=2). C, Actin stress fiber formation in response to thrombin in cells transducing GFP-tagged C1 or GFP-tagged C1-S96A mutant. Cells were stimulated with thrombin for 5 min and fixed followed by staining with Alexa-labeled phallodin to determine actin stress fiber formation by confocal imaging. D, Cells plated on gold electrodes were transduced with C1- or C1-S96A-GDI-1 mutant to measure the time course of changes in transendothelial electrical resistance (TER) after addition of 50 nM thrombin. Data represent means +S.E.M of changes in TER from multiple experiments. TER values are calculated as change in resistance over the value at time zero (n=7). E, Means +S.E.M of changes in TER in HPAE cells transducing C1- or C1-S96A GDI-1 mutant following pretreatment with Go6976. Cells plated on gold electrodes were transduced with C1 or S96A-GDI-1 mutant. After 30 hrs cells were pretreated with 1 uM Go6976 to inhibit PKCα for 30 min and changes in transendothelial electrical resistance (TER) after addition of 50 nM thrombin were determined. TER values are calculated as change in resistance over the value at time zero (n=8).

### DETAILED DESCRIPTION OF THE INVENTION

A. Definitions.

“Inhibit” or “down-regulate” means that the expression of a target gene, or level of RNAs or equivalent RNAs encoding one or more proteins or isoforms, or activity of one or more proteins is reduced below that observed in the absence of the compositions of the invention.

By “up-regulate” is meant that the expression of the gene, or level of RNAs or equivalent RNAs encoding one or more proteins or isoforms, or activity of one or more proteins, such as an inhibiter of GDI-1, is greater than that observed in the absence of the compositions of the invention.

By “modulate” it is meant that the expression of the gene, or level of RNAs or equivalent RNAs encoding one or more protein subunits, or activity of one or more proteins or protein isoforms is up-regulated or down-regulated, such that the expression, level, or activity is greater than or less than that observed in the absence of the compositions of the invention.

By “gene” it is meant a nucleic acid that encodes an RNA, for example, nucleic acid sequences including but not limited to structural genes encoding a polypeptide.

“Complementarity” refers to the ability of a nucleic acid to form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, antisense or triple helix inhibition. Determination of binding free energies for nucleic acid molecules is well known in the art (see, e.g., Turner et al., 1987, CSH Symp. Quant. Biol. LII pp. 123 133; Frier et al., 1986, Proc. Nat. Acad. Sci. USA 83:9373 9377; Turner et al., 1987, J. Am. Chem. Soc. 109:3783 3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

The administration of the herein described agents to a patient can be intravenous, intraarterial, intraperitoneal, intramuscular, subcutaneous, intrapleural, intrathecal, by perfusion through a regional catheter, or by direct intralesional injection. When administering these nucleic acid molecules by injection, the administration may be by continuous infusion, or by single or multiple boluses. The dosage of the administered agent will vary depending upon such factors as the patient's age, weight, sex, general medical condition, and previous medical history. Typically, it is desirable to provide the recipient with a dosage of a nucleic acid agent which is in the range of from about 1 pg/kg to 10 mg/kg (amount of agent/body weight of patient), although a lower or higher dosage may also be administered.

A composition is said to be a “pharmaceutically acceptable carrier” if its administration can be tolerated by a recipient patient. Sterile phosphate-buffered saline is one example of a pharmaceutically acceptable carrier. Other suitable carriers are well-known in the art. See, for example, REMINGTON'S PHARMACEUTICAL SCIENCES, 18^{th} Ed. (1990).

For purposes of immunotherapy, an immunoconjugate and a pharmaceutically acceptable carrier are administered to a patient in a therapeutically effective amount. A combination of an immunoconjugate and a pharmaceutically acceptable carrier is said to be administered in a “therapeutically effective amount” if the amount administered is physiologically significant. An agent is physiologically significant if its presence results in a detectable change in the physiology of a recipient patient.

Additional pharmaceutical methods may be employed to control the duration of action of an immunoconjugate in a therapeutic application. Control release preparations can be prepared through the use of polymers to complex or adsorb an immunoconjugate. For example, biocompatible polymers include matrices of poly(ethylene-co-vinyl acetate) and matrices of a polyanhydride copolymer of a stearic acid dimer and sebacic acid. Sherwood et al., Bio/Technology 10: 1446-1449 (1992). The rate of release of an agent from such a matrix depends upon the molecular weight of the molecule, the amount of molecule within the matrix, and the size of dispersed particles. Saltzman et al., Biophysical. J. 55:163-171 (1989); and Sherwood et al., Bio/Technology 10:1446-1449 (1992). Other solid dosage forms are described in REMINGTON'S PHARMACEUTICAL SCIENCES, 18^{th} Ed. (1990).

B. Screening Systems and Methods of Treatment.

One aspect of the invention provides a method for treating a subject that has or is at risk of developing an inflammatory disease. The method comprises administering to the subject a composition comprising an agent that modulates an oncogenic transcription factor activity. In one embodiment, the agent decreases the protein activity or protein level of GDI-1. In another embodiment, the agent decreases the mRNA level of GDI-1. In yet another embodiment, the agent is selected from the group consisting of hormones, cytokines, small molecules, antibodies, antisense oligonucleotides, chemicals, and RNA inhibitors. In some embodiments, the subject being treated has or is at risk of developing an inflammatory disease.

In another aspect of the present invention, a method is provided for screening for an agent that modulates the activity of GDI-1. The method comprises exposing a sample to an agent to be tested, detecting a level of activity of GDI-1 and comparing the level of activity of GDI-1 to a control level.

In another aspect, the invention provides a method for decreasing inflammation. The method comprises contacting a cell in vitro or in vivo with a composition comprising an agent that modulates GDI-1 activity. In one embodiment, the agent modulates GDI-1 activity, for example, by decreasing the mRNA level of GDI-1. In another embodiment, the agent is selected from the group consisting of hormones, cytokines, small molecules, antibodies, antisense oligonucleostides, chemicals, and RNA inhibitors.

In one general aspect, the present invention provides an assay method for a substance with ability to modulate, e.g. disrupt or interfere with interaction or binding between GDI-1 and a kinase (such as PKCα), the method including:

(a) bringing into contact a substance according to the invention including a peptide fragment of GDI-1 or a derivative, variant or analogue thereof as disclosed, a substance including the relevant fragment of a kinase or a variant, derivative or analogue thereof, and a test compound, under conditions wherein, in the absence of the test compound being an inhibitor of interaction or binding of the substances, the substances interact or bind; and (b) determining interaction or binding between the substances.

A test compound which disrupts, reduces, interferes with or wholly or partially abolishes binding or interaction between said substances (e.g. including a GDI-1 fragment and including a PKCα fragment), and which may modulate GDI-1 and/or PKCα activity, may thus be identified.

Phosphorylation may be determined for example by immobilizing GDI-1 or a fragment, variant or derivative thereof, e.g. on a bead or plate, and detecting phosphorylation using an antibody or other binding molecule which binds the relevant site of phosphorylation with a different affinity when the site is phosphorylated from when the site is not phosphorylated. Such antibodies may be obtained by means of any standard technique as discussed elsewhere herein, e.g. using a phosphorylated peptide (such as a fragment of GDI-1). Binding of a binding molecule which discriminates between the phosphorylated and non-phosphorylated form of GDI-1 or relevant fragment, variant or derivative thereof may be assessed using any technique available to those skilled in the art, which may involve determination of the presence of a suitable label, such as fluorescence. Phosphorylation may be determined by immobilisation of GDI-1 or a fragment, variant or derivative thereof, on a suitable substrate such as a bead or plate, wherein the substrate is impregnated with scintillant, such as in a standard scintillation proximetry assay, with phosphorylation being determined via measurement of the incorporation of radioactive phosphate. Rather than immobilizing GDI-1, its phosphorylation by a kinase, such as PKCα, may be assayed by means of allowing its radio- or other labeling in solution, with a suitable specific binding member such as an antibody or a GDI-1 -binding fragment thereof being used to pull it out for determination of labeling. Phosphate incorporation into GDI-1 or a fragment, variant or derivative thereof, may be determined by precipitation with acid, such as trichloroacetic acid, and collection of the precipitate on a suitable material such as nitrocellulose filter paper, followed by measurement of incorporation of radiolabeled phosphate. SDS-PAGE separation of substrate may be employed followed by detection of radiolabel.

Recombinant expression vectors may be constructed by incorporating nucleotide sequences within a vector according to methods well known to the skilled artisan. A wide variety of vectors are known that are useful in the invention. Suitable vectors include plasmid vectors and viral vectors, including retrovirus vectors, adenovirus vectors, adeno-associated virus vectors and herpes viral vectors. The vectors may include other known genetic elements necessary or desirable for efficient expression of the nucleic acid in a specified host cell, including regulatory elements. For example, the vectors may include a promoter and any necessary enhancer sequences that cooperate with the promoter to achieve transcription of the gene. The nucleotide sequence may be operably linked to such regulatory elements.

Such a nucleotide sequence is referred to as a “genetic construct.” A genetic construct may contain a genetic element on its own or in combination with one or more additional genetic elements, including but not limited to genes, promoters, or enhancers. In some embodiments, these genetic elements are operably linked.

C. Mechanism for GDI-1 Regulation of RhoA

GDI-1, by binding to RhoGTPases, restricts their activation by GEFs and thereby plays a primary role in regulating downstream RhoGTPase-mediated signaling (5, 8, 26). Structural analysis showed that both N- and C-terminus domains of GDI-1 may contribute to regulating its inhibitory activity on RhoGTPases (10-12, 14). Post-translational modification of GDI-1 and GDI-1 interactions with ERMs proteins as well as phospholipids can modulate GDI-1's inhibitory activity (3, 4, 6, 13). We have shown that thrombin induces PKCα-dependent GDI-1 phosphorylation resulting in RhoA activation (23), indicating an important functional role of phosphorylation of GDI-1 in the mechanism of RhoA activation. We extended these findings by identifying the specific phosphorylation sites of GDI-1 required to selectively regulate RhoA activity and its functional significance in the mechanism of RhoA-mediated increase in endothelial permeability.

Below, we show that thrombin failed to induce RhoA activation and actin stress fiber formation in endothelial cells transduced with full-length (FL)-GDI-1. These findings also demonstrate that FL-GDI-1 plays a central role in regulating RhoA activity downstream of the Gαq and Gα12/13 heterotrimeric G proteins. Consistent with the findings of this study, microinjection of FL-GDI-1 protein in fibroblasts or MDCK cells also inhibited RhoA-induced cell motility, formation of stress fibers, and development of focal adhesions (17, 30-33). Since GDI-1 binds RhoA (5, 8, 26) the expression of FL-GDI-1 in endothelial cells by sequestering RhoA may have prevented the thrombin-induced RhoA signaling in endothelial cells. In another study, we used V14RhoA mutant which contains all the features of wild-type RhoA except mutation of Gly14 to Va1 renders it ineffective to GTPase activity (9, 25, 27). However, V14RhoA is able to bind to its effectors and activate downstream signaling (9, 25, 27). We observed that FL-GDI-1 expression also prevented the signaling induced by expression of this constitutive-active RhoA mutant, demonstrating the ability of the FL-Rho-GDI-1 to bind to the active RhoA and hold it in abeyance.

PKCα is known to phosphorylate GDI-1 and thus may regulate GDI function (23). Based on the finding that phosphorylation of GDI-1 can alter its inhibitory activity to RhoGTPases (6), we examined phosphorylation of the C terminus (aa 69-204) as induced by PKCα. We observed that PKCα was capable of directly phosphorylating GDI-1 C terminus. However, expression of this domain in endothelial cells prevented RhoA activation induced by thrombin. Thus, the C-terminus of GDI-1 retained its inhibitory activity on RhoA. Deletion analysis of the C-terminus was carried to address further the role of GDI-1 phosphorylation in regulating RhoA activity. The inhibitory activity of GDI-1 C-terminus on RhoA activity was lost upon deletion of the C-terminus into C1 (aa 69 to 140) and C2 (aa 69 to 141) components. Co-expression of kinase defective PKCα along with either C1 or C2 rescued the thrombin-induced inhibitory activity of these subunits on RhoA, suggesting that phosphorylation of C1 and C2 is important in the mechanism of RhoA activation. It is possible that phosphorylation sites in transduced C1 and C2 constituents are unmasked, thereby preventing inhibitory activity C terminus on RhoA.

We identified that phosphorylation of Ser96 residue on C1-GDI-1 mutant plays a critical role in regulating RhoA activation. Mutation of Ser96 to alanine of the GDI-1 C1-terminus prevented thrombin-induced RhoA activation in association with absence of phosphorylation of the C-terminus. In contrast, mutation of Thr197 did not prevent the thrombin-induced phosphorylation of the C-terminus and RhoA activation induced by thrombin. The effect of mutation of S176 was intermediate in that there was a partial reduction in thrombin-induced phosphorylation of C terminus as well as decreased RhoA activation.

Because GDI-1 binds to RhoA, Rac1 and Cdc42, it is believed to be a nonspecific inhibitor of activation of these GTPases (5, 7, 8). However, our evidence indicates otherwise. We observed that phosphorylation of GDI-1 on C-terminus provides specificity for GDI-1 inhibition of RhoGTPase activity. Phosphorylation of Ser96 was sufficient to induce the activation of RhoA but not Rac or Cdc42. The mechanism by which the phosphorylation of GDI-1 at Ser96 modifies the ability of GDI-1 to specifically regulate RhoGTPase activity is unclear. A negative charge conferred by the phosphorylated residue may facilitate the release of GTPases from RhoGTPase-GDI-1 complex (5, 8, 26). Thus, it is possible that charge differences attributed to one serine (Ser96) may determine the specificity of RhoA release from the GDI-1-RhoGTPase complex. We also showed that in addition to GDI-1, thrombin also phosphorylates p115RhoGEF via PKCα (16). This finding in the context of the present observations suggests that the binding sites for GDI-1 or GEFs on RhoGTPases may not be mutually exclusive (1, 8). Therefore, co-incident phosphorylation of both p115RhoGEF and GDI-1 by PKCα may be required for full RhoA activation.

As RhoA activation is known to induce endothelial barrier dysfunction, a hallmark of inflammatory diseases such as acute lung injury (22), we addressed whether S96 of GDI-1 has a functional role in regulating increased endothelial permeability response to thrombin challenge (16, 22, 23). We observed that S96A GDI-1 mutant prevented actin stress fiber formation and significantly reduced endothelial barrier dysfunction induced by thrombin. Moreover, the reduction in endothelial permeability observed in S96A GDI-1 mutant-expressing cells was recapitulated following inhibition of PKCα activity in endothelial cells transducing the C1-GDI-1 mutant. These studies demonstrate that PKCα-induced GDI-1 phosphorylation at Ser96 is a requirement for RhoA-induced disruption of the endothelial barrier. In this regard, drugs targeting S96 of GDI-1 may represent a novel class of anti-inflammatory therapeutics capable of preventing increased endothelial permeability in response to RhoA activation.

The below-identified results indicate that GDI-1 C-terminus phosphorylation at Ser96 reduces the affinity of GDI-1 for RhoA, and thereby selectively induces RhoA activation. Therefore, phosphorylation of Ser96 of the GDI-1 C-terminus represents a novel target for regulating RhoA activity and RhoA-dependent increase in endothelial permeability associated with inflammatory diseases.

Having now generally described the invention, the same will be more readily understood through reference to the following Examples which are provided by way of illustration, and are not intended to be limiting of the present invention.

### EXAMPLE 1

### Materials and Protocols Used in Below-Identified Examples 2-5

Materials

Human pulmonary arterial endothelial (HPAE) cells and endothelial growth medium (EBM-2) were obtained from Clonetics (San Diego, Calif.). Human α-thrombin was obtained from Enzyme Research Laboratories (South Bend, Ind.). Superfect and DEAE-dextran transfection reagents were purchased from Qiagen (Valencia, Calif.) whereas Lipofectamine 2000 transfection reagent was obtained from Invitrogen (Carlsbad, Calif.). Nucleofector HCAEC kit and electroporation system were from Amaxa (Gaithersburg, Md.). Alexa-phallodin, DAPI, ProLong Gold antifade were from Molecular Probes (Eugene, Oreg.). Glutathione sepharose 4B and ³²P-y-ATP were purchased from Amersham Biosciences (Piscataway, N.J.). Electrodes for trans-endothelial resistance measurements were obtained from Applied Biosciences (Troy, N.Y.). Anti-green fluorescent protein (GFP), anti-Myc, and HRP-conjugated anti-mouse IgG antibodies and protein A/G beads were purchased from Santa Cruz Biotechnology (San Diego, Calif.). Anti-RhoA, Cdc42 and Rac1 antibody were purchased from BD Biosciences (San Rose, Calif.). Fc-fragment specific horseradish peroxidase (HRP)-conjugated anti-mouse IgG antibody was purchased from Jackson Immuno Research Laboratories (West Grove, Pa.). Recombinant PKCα, GST-rhotekin-Rho binding domain beads and PAK (p21-activated kinase) PBD-GST beads were purchased from Cytoskeleton (Denver, Colo.).

Endothelial cell culture: HPAE cells were cultured in a T-75 flask coated with 0.1% gelatin in EBM-2 medium supplemented with 10% fetal bovine serum and maintained at 37° C. in a humidified atmosphere of 5% CO₂ and 95% air until they formed a confluent monolayer. Cells from each of the primary flasks were detached with 0.05% trypsin, containing 0.02% EDTA for indicated experiments. In all experiments, unless otherwise indicated a confluent monolayer of HPAE cells was washed twice with serum-free, MCDB-131 medium and incubated in the same serum-free medium for 1 h before treatment with thrombin. In all experiments, HPAE cells between passages 5 and 8 were used.

Construction of GFP tagged GDI-1 mutants: Various RhoGDI-1 fragments were generated by PCR amplification (PFU; Stratagene) using the cDNA clone pOTB7-GDI-1 as a template. Agarose gel purified PCR fragments were digested with EcoRI/BamHI or EcoRI/Xho I restriction enzymes and cloned into pEGFP-C3 vector. All subsequent clones were sequenced to ensure sequence integrity. Clone specific primer pairs are as listed; pEGFP-FL-GDI-1 mutant (AA1-204) forward primer 5′-AGGAATTCGAATGGCTGAGCAGGAGCCCAC-3′ and reverse primer 5′-CGGGATCCTCATCAGTCCTTCCAGTCCTTC-3′; pEGFP-C-GDI-1 mutant (AA69-204), forward primer 5′ AGGAATTCGAAACGTCGTGGT GACTGG 3′ and reverse primer 5′ CGGGATCCTCATCAGTCCTTCCAGTCCTTC 3′; pEGFP-C1-GDI-1 mutant (AA 69-114) forward primer 5′-AGGAATTCGAAACGTCGTGGTGACTGG-3′ and reverse primer 5′-CGGGATCCTCATCAGTCAATCTTGACGCCTTTCC-3′; pEGFP-C2-GDI-1 (AA 115-204) forward primer 5′-AGGAATTCGAAAGACTGACTACATGGTAGGC-3′ and reverse primer 5′-CGGGATCCTCATCAGTCCTTCCAGTCCTTC-3′; Phosphorylation defective mutants were generated through a two-step PCR process. In short, over-lapping DNA fragments containing the base pair changes were generated in separate PCR reactions during the first round of PCR using the cDNA clone pOTB7-GDI as a template. These fragments were combined and used as the second round PCR template to amplify the entire GDI cDNA containing the specific amino acid changes. Agarose gel purified PCR fragments were digested with EcoRI/BamHI restriction enzymes and cloned into pEGFP-C3. Subsequent clones were sequenced to ensure sequence integrity. Clone specific primer pairs with amino acid change(s) in bold are as listed; pEGFP-S96A C1-GDI-1—first round primer pairs—forward primer (1) 5′-AGGAATTCGAATGGCTGAGCAGGAGCCCAC-3′ and reverse primer (1) 5′-TTCTTGAAGGCCTCCAGGTCGC-3′ and forward primer (2) 5′-ACCTGGAGGCCTTCAAGAAGC-3′ and reverse primer (2) 5′-CGGGATCCTCATCAGTCCTTCCAGTCCTTC-3′. The second round PCR used primer pair forward primer (1) and reverse primer (2). pEGFP-S176A-GDI-1—first round primer pairs—forward primer (1) 5′-AGGAATTCGAATGGCTGAGCAGGAGCCCAC-3′ and reverse primer (1) 5′-GCGGGACTTGATGGCGTAGC-3′ and forward primer (2) 5′-AGCTACGCCATCAAGTCCCGC-3′ and reverse primer (2) 5′-CGGGATCCTCATCAGTCCTTCCAGTCCTTC-3′. The second round PCR used primer pair; forward primer (1) and reverse primer (2). pEGFP-T197A-GDI-1—first round primer pairs—forward primer (1) 5′-AGGAATTCGAATGGCTGAGCAGGAGCCCAC-3′ and reverse primer (1) 5′-TCCTTCTTGATGGCGAGATTCC-3′ and forward primer (2) 5′-AATCTCGCCATCAAGAAGGAC-3′ and reverse primer (2) 5′-CGGGATCCTCATCAGTCCTTCCAGTCCTTC-3′. The second round PCR used primer pair; forward primer (1) and reverse primer (2).

Cell transfection: cDNA was purified using the Endo-free Qiagen kit. cDNA was transduced into cells by electroporation or using Superfect transfection reagent. HPAE cells grown up to 70% confluency were trypsinized, mixed with 4 μg of cDNA along with 100 ul HCAEC nucleofector solution. Cells were rapidly electroporated by Amaxa nucleofector device using manufacturer's recommended program (S-05) dedicated for human coronary endothelial cells. The cells were removed, mixed in EBM-2 and plated 60 mm dishes. HPAE cells plated on electrodes were transfected with indicated with indicated cDNA using Superfect transfection reagent following manufacture protocol. The cells were used after 24 hr transfection when there was evidence of the expression of protein. COS-7 cells were cultured in Dulbecco's modified Eagle's medium (DMEM) supplemented with 10% fetal bovine serum. COS-7 cells were transfected with appropriate cDNA using Lipofectamine 2000 following manufacturer protocol.

Seum response element reporter gene activity: Transfections were performed using the DEAE-dextran method as described (23). Following serum deprivation, cells were stimulated with 50 nM of a-thrombin for 5 hr. Cell extracts were prepared and assayed for luciferase activity using the Dual Luciferase Reporter Assay System (Promega). SRE-luciferase activity was expressed as the ratio of firefly and renilla luciferase activity.

Actin stress fiber staining: Following stimulation with thrombin, cells were fixed and incubated with Alexa-labeled phalloidin to stain stress fibers. Cells viewed with a 63×1.2 NA objective using a Zeiss LSM 510 confocal microscope (16).

Measurements of RhoGTPase activity: HPAE cells transducing indicated cDNA were stimulated with 50 nM thrombin. RhoA, activity was measured using GST-rhotekin-Rho binding domain whereas GST-PAK binding domain was used to quantify Rac or Cdc42 activities as described (16, 23).

Transendothelial resistance measurement: The time course of endothelial cell retraction in real time, a measure of increased endothelial permeability, was measured according to the procedure described previously (16, 23).

Immunoprecipitation: HPAE cells or COS-7 cells were washed with PBS and lysed using radioimmune precipitation assay buffer (1% Triton-X, 150 mM NaCl, 10 mM Tris, 1 him EDTA, 1 mM EGTA, 1 mM Na₃VO₄, 1 mM phenylmethylsulfonyl fluoride, 0.5% Nonidet P-40, and 2 μg/ml each of pepstatin A, leupeptin, and aprotinin) (16, 23). The lysate was scraped and cleared by centrifugation at 4° C. at 14,000×g for 10 min. Cell lysate was then precleared with protein A/G-agarose beads for 30 minutes, and the proteins were immunoprecipitated with the appropriate antibody overnight at 4° C. followed by incubation with protein A/G-agarose for 6 h at 4° C. The beads were then collected by centrifugation and washed 3× with detergent-free radioimmune precipitation assay buffer.

In vitro kinase assay: COS 7 cells transducing various GDI mutants were lysed and immunoprecipitated with anti-GFP antibody. The beads were incubated with purified PKCα to determine phosphorylation as described (23).

Statistical analysis: Comparisons between experimental groups were made by ANOVA and t-test, using SigmaStat software. Differences in mean values were considered significant at p <0.05.

### EXAMPLE 2

### GDI-1 Expression Prevents SRE Activation and Actin Stress Fiber Formation Induced by Thrombin

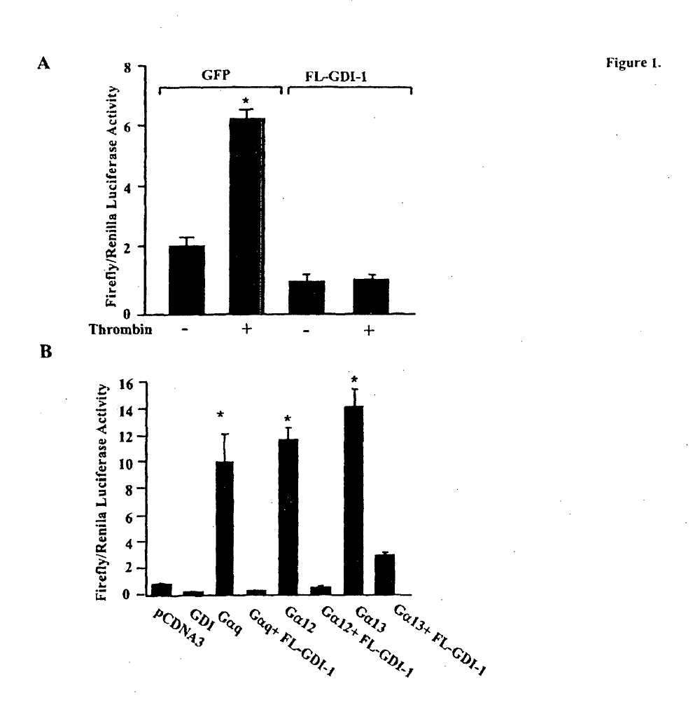

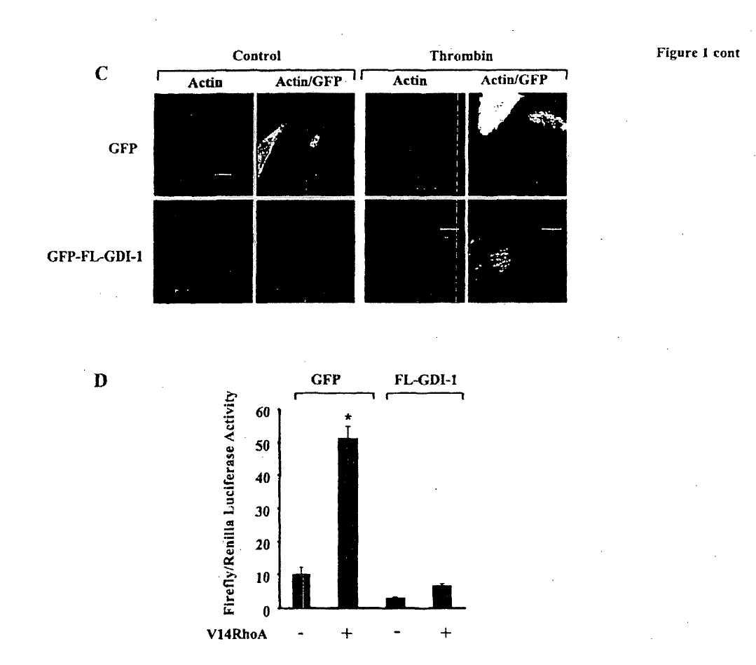

We previously showed that RhoA activity is required for thrombin-induced stimulation of serum response element (SRE) activity in endothelial cells (16, 23). In the present experiments, we measured SRE activation to determine the role of full-length (FL) GDI-1 in regulating RhoA-induced SRE activity. HPAE cells transfected with control vector or vector containing FL-GDI-1 mutant were stimulated with thrombin after which SRE activity was determined. Thrombin induced a marked SRE activation in GFP-transducing HPAE cells whereas this response was not seen in HPAE cells transduced with FL-GDI-1 mutant (FIG. 1A).

Because Gα₁₂, Gα₁₃ and Gα_{q} heterotrimeric G-proteins contribute to RhoA activation (2, 16, 19, 24, 34), we addressed the role of FL-GDI-1 in regulating SRE activation induced by expression of Gα_{q}, Gα₁₂, or Gα₁₃. We found increased SRE activity in HPAE cells transducing constitutively active mutants of Gα_{q}, Gα₁₂, or Gα₁₃. Co-expression of Gα_{q}, Gα₁₂, or Gα₁₃ with GDI-1 prevented SRE activation by these G-proteins (FIG. IB). Thus, GDI-1 plays a central role in inducing SRE activation downstream of Gα_{q} as well as Gα₁₂ and Gα₁₃ proteins.

Next we determined the effects of GDI-1 expression on SRE activation induced by transduction of constitutively active RhoA (V14RhoA). In comparison with vector, HPAE cells transducing V14RhoA induced a 7-fold increase in SRE reporter gene activity (FIG. 1C). However, co-expression of FL-length GDI-1 prevented the V14RhoA-induced SRE activation. As RhoA activation is known to regulate actin stress fibers formation, we also determined alterations in actin stress fibers in response to thrombin in GFP- or GFP-GDI-1-transducing HPAE cells. Confocal images show that thrombin stimulated actin stress fiber formation in cells transduced with control vector whereas the response was not observed in cells transducing FL-GDI-1 (FIG. 1D). These findings demonstrate that exogenous GDI-1 prevents the thrombin-induced RhoA-dependent signaling and actin stress fiber formation in endothelial cells.

### EXAMPLE 3

### PKCα-Induced GDI-1 Phosphorylation Signals RhoA in Response to Thrombin

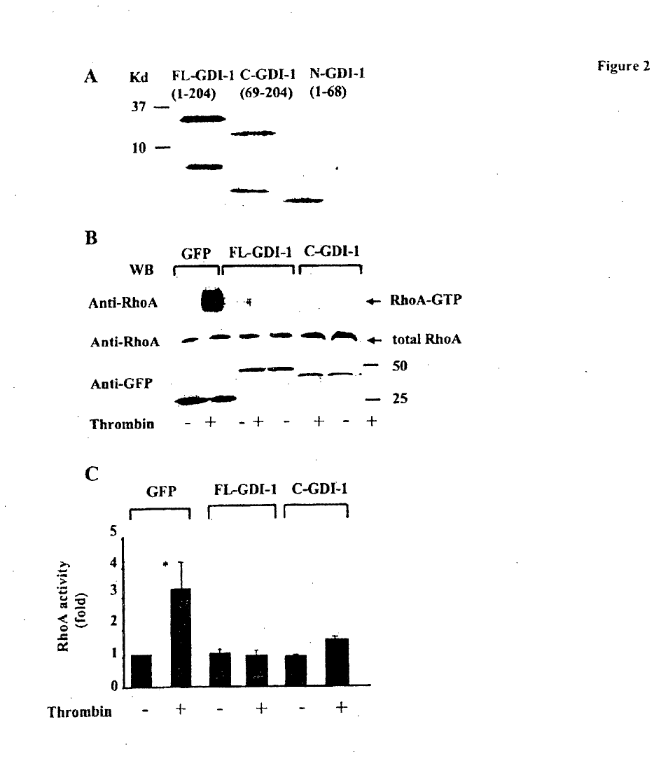

GDI-1 is composed of a flexible 69 amino acid long N-terminus and a folded 135 amino acid long C-terminus (5, 8, 26). We have shown that thrombin-induced GDI-1 phosphorylation by PKCα can signal activation of RhoA (23). To address the possibility that GDI-1 phosphorylation may regulate the inhibitory activity of GDI-1 on RhoGTPases (6) we first identified the region that is phosphorylated by PKCα. We used purified FL-GDI-1, GDI-1 N-terminus (1-68) or GDI-1 C-terminus (69-204) proteins and incubated with PKCα in vitro. We found that PKCα phosphorylated the C-terminus (FIG. 2A). Thus, we expressed Fl-GDI-1 or GDI-1 C-terminus in HPAE cells and used rhotekin bound fusion proteins to determine whether phosphorylation of GDI-1 alters GDI-1 inhibition of RhoA activity. As shown in FIG. 2B, thrombin failed to induce RhoA activity in endothelial cells transducing FL-length-GDI-1 (FIG. 2B-C). Surprisingly, thrombin also failed to induce RhoA activation in endothelial cells transducing C-terminus domain of GDI-1 (FIG. 2B-C).

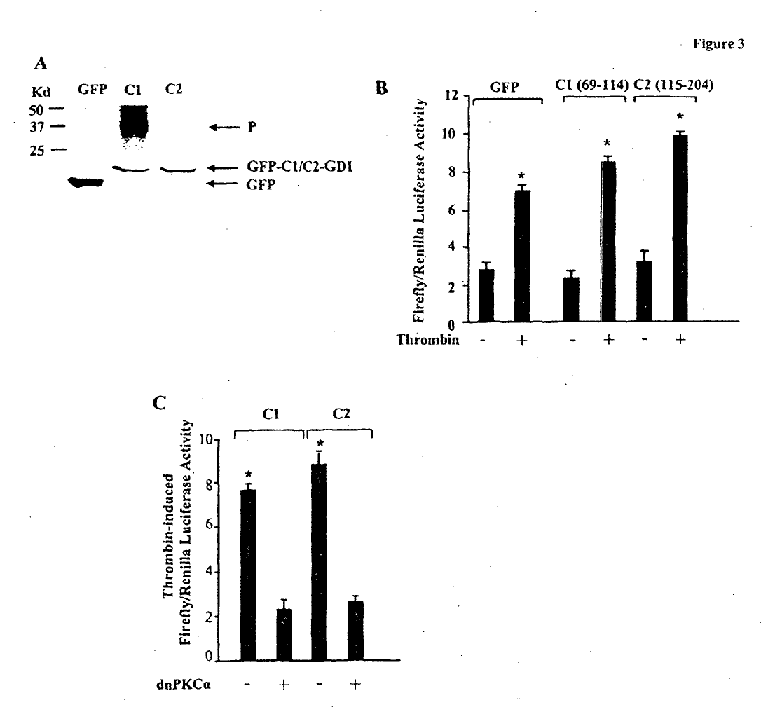

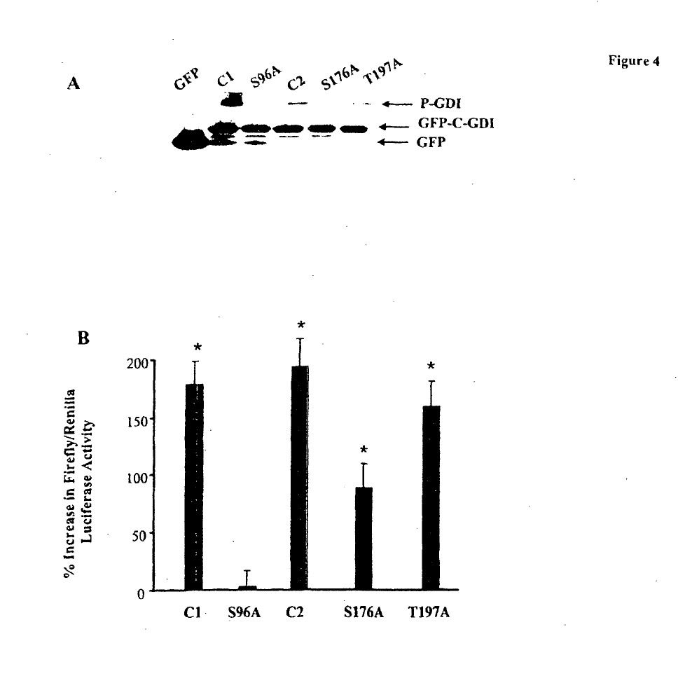

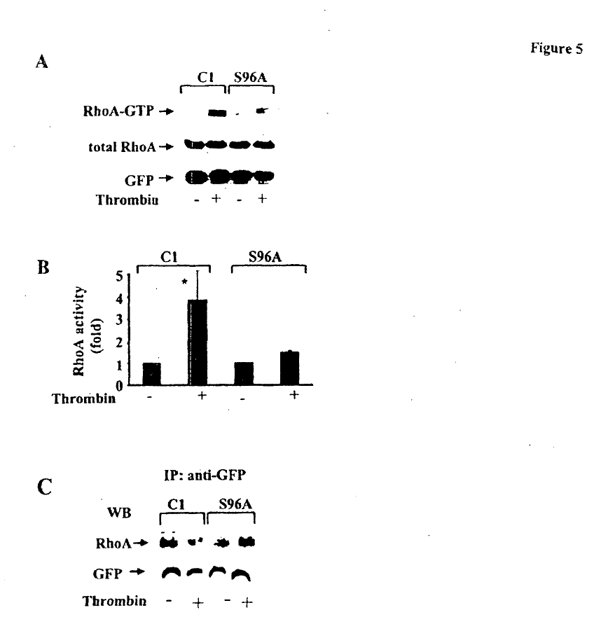

To address the possibility that intra-molecular interactions between the C-terminus domain of GDI-1 may have altered its inhibitory effect on RhoA, we deleted the C-terminus to GFP-tagged generate C1 (aa 69-140) and C2 (aa 141-204) mutants. We expressed GFP-tagged C1- or C2-GDI-1 mutants in COS-7 cells to determine if truncation effected the PKCα phosphorylation of C-terminus. Lysates were COS-7 cells were immunoprecipitation with anti-GFP antibody and these immunocomplexes were used for in vitro kinase assay. As shown in FIG. 3A, PKCα markedly phosphorylated the C1 domain of GDI-1 as compared to C2 domain. Next, we transduced these mutants in HPAE cells to determine their effects on thrombin-induced SRE activation (FIG. 3B). We observed that further truncation of full-length C-terminus prevented its inhibitory activity on RhoA; that is the thrombin-induced SRE activation was not affected in HPAE cells transducing C1 or C2 GDI-1 mutants. We also co-transduced C1- or C2-GDI mutants with kinase-defective PKCα mutant to determine whether the C-terminus-induced inhibition of RhoA activity can be rescued. As shown in FIG. 3C, co-expression of kinase-defective PKCα mutant with C1 or C2 domain of GDI-1 in HPAE cells prevented thrombin-induced SRE generation. Using NetPhos program that predicts the phosphobases in GDI-1, we found that the C-terminus of GDI-1 contains consensus PKCα phosphorylation sites at Ser96, Ser176, and Thr197 residues. To investigate whether PKCα phosphorylates GDI-1 at these sites, we mutated the serine or threonine residues with alanine to generate non phosphorylatable S96A, S176A, and T197A GDI-1 mutants. As shown in FIG. 4A, PKCα failed to phoshorylate S96A GDI-1 mutant whereas phosphorylation of C2 fragments containing S176A or S197A mutation was reduced (FIG. 4A).

To investigate the causal role of PKCα-induced phosphorylation on C-terminus in regulating RhoA activity, HPAE cells transducing phosphor-defective S96A, S176A, and T197A GDI-1 mutants were stimulated with thrombin to determine SRE and RhoA activity. We found that transduction of only the S96A GDI-1 mutant in HPAE cells was sufficient to prevent SRE activation in response to thrombin (FIG. 4B). Furthermore, thrombin-induced RhoA activation was markedly suppressed in HPAE cells transducing the S96A GDI-1 mutant (FIG. 5A-B). These findings demonstrate that phosphorylation of Ser96 of Rho-GDI-1 is the primary determinant of RhoA activation.

We next addressed the possibility that phosphorylation at Ser96 residue enables RhoA activation by inducing the dissociation of RhoA from GDI-1 -RhoA complex. HPAE cells transducing C1 or S96A-C1 mutant were stimulated with thrombin and lysates were immunoprecipitated with anti-GFP Ab followed by Western blotting with anti-RhoA Ab. We found that thrombin increased the dissociation of RhoA from C1-GDI-1 (FIG. 5C). However, this effect was not seen in cells transducing S96A C1 mutant (FIG. 5C). Thus, phosphorylation of GDI-1 at serine 96 residue is crucial in decreasing its binding affinity for RhoA, thereby inducing RhoA activation.

### EXAMPLE 4

### Phosphor-Defective S96A-GDI-1 Mutant Fails to Prevent Rac1 or Cdc42 Activity

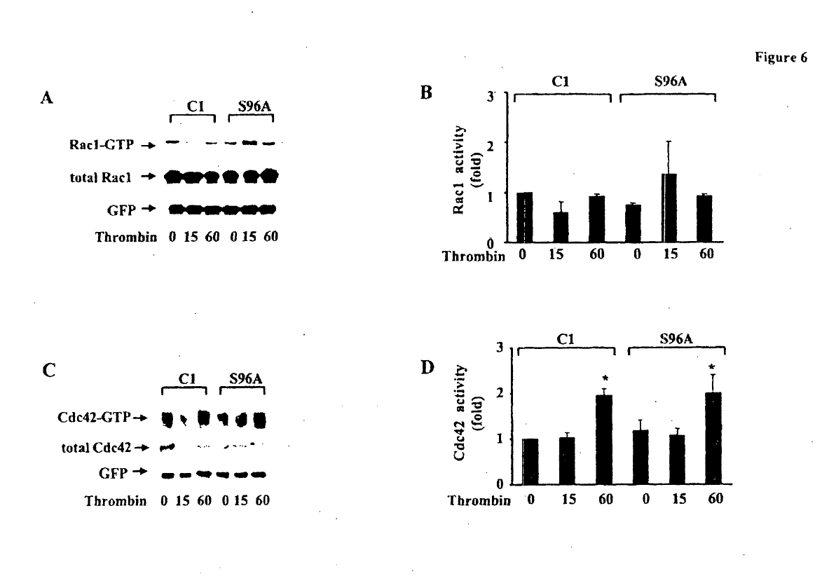

In endothelial cells, thrombin is known to induce a transient activation of RhoA (15, 22). Under these conditions, Rac1 was shown to be inactivated (18, 35) whereas thrombin induces a delayed activation of Cdc42 (18). We determined Rac1 and Cdc42 activities in HPAE cells transducing S96A GDI-1 mutant to assess whether mutation on serine 96 residue also alters the activation of these GTPases in response to thrombin. As shown in FIG. 6A-D, expression of S96A-GDI-1 mutant in HPAE cells had no effect on thrombin-induced alterations in Rac1 and Cdc42 activities (FIG. 6A-D). Thus, the S96A GDI-1 mutant selectively prevented RhoA activation.

### EXAMPLE 5

### Phosphor-Defective S96A Mutant Inhibits Actin Stress Fiber Formation and Loss of Endothelial Barrier Function Induced by Thrombin

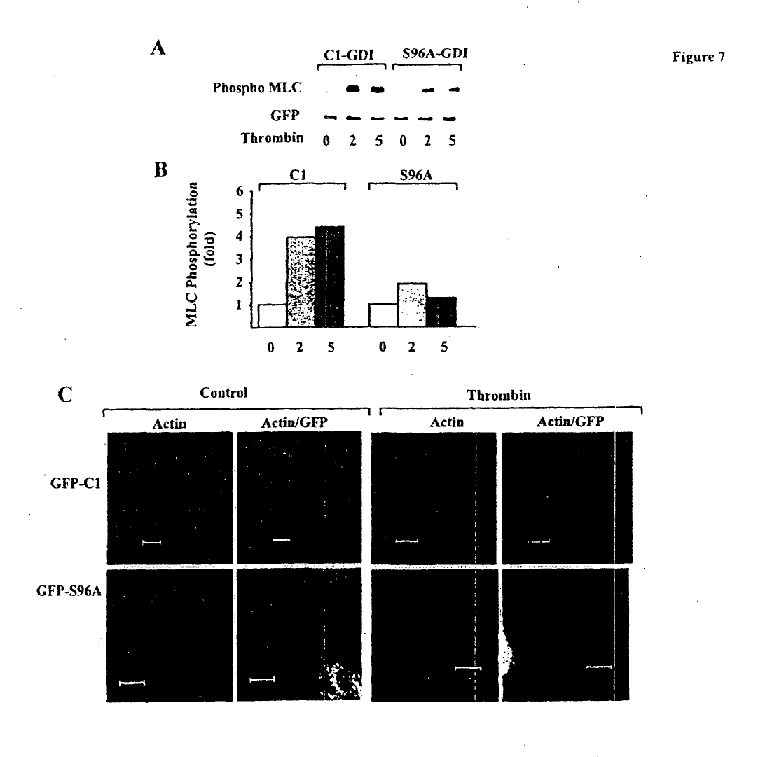

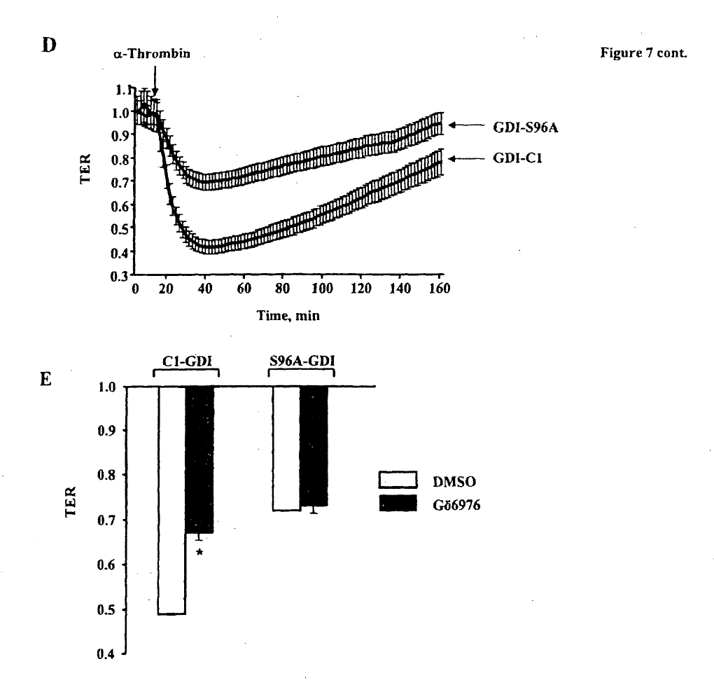

Because RhoA activation increases endothelial permeability by inducing MLC phosphorylation and actin stress fiber formation (22), we determined the requirement of GDI-1 phosphorylation at Ser96 residue on thrombin-induced actin stress fiber formation and MLC phosphorylation. We also determined transendothelial electrical resistance (TER) in endothelial monolayers to address the functional role of S96A of GDI-1 in regulating endothelial barrier function. As shown in FIG. 7A-C, thrombin induced MLC phosphorylation as well as increased stress fiber formation in cells transfected with C1-GDI-1 mutant; however, this response was markedly decreased in cells transducing S96A GDI-1 mutant (FIG. 7A-C). We also observed that expression of S96A GDI-1 mutant significantly reduced the thrombin-induced decrease in TER (FIG. 7D). To corroborate that these results with the effects of PKCα-mediated downregulation of GDI-1 function, we inhibited PKCα using Gö976 (16). Pretreatment with G66976 significantly reduced the thrombin-induced decrease in TER in HPAE cells transducing C1-GDI-1 mutant but this response was not seen in cells transducing S91A GDI-1 mutant (FIG. 7E). These findings show that PKCα phosphorylation of GDI-1 at serine 96 residue plays an essential role in thrombin-induced RhoA activation and signaling the loss of endothelial barrier function.

It is understood that the disclosed invention is not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a host cell” includes a plurality of such host cells and equivalents thereof known to those skilled in the art, and so forth.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. Publications cited herein and the material for which they are cited are specifically incorporated by reference. Nothing herein is to be construed as an admission that the invention is not entitled to antedate such disclosure by virtue of prior invention.

### REFERENCES

1. Biou, V., and J. Cherfils. 2004. Structural principles for the multispecificity of small GTP-binding proteins. Biochemistry 43:6833-40.

2. Chikumi, H., J. Vazquez-Prado, J. M. Servitja, H. Miyazaki, and J. S. Gutkind. 2002. Potent activation of RhoA by Galpha q and Gq-coupled receptors. J Biol Chem 277:27130-4.

3. Chuang, T. H., B. P. Bohl, and G. M. Bokoch. 1993. Biologically active lipids are regulators of Rac.GDI complexation. J Biol Chem 268:26206-11.

4. Chuang, T. H., X. Xu, U. G. Knaus, M. J. Hart, and G. M. Bokoch. 1993. GDP dissociation inhibitor prevents intrinsic and GTPase activating protein-stimulated GTP hydrolysis by the Rac GTP-binding protein. J Biol Chem 268:775-8.

5. DerMardirossian, C., and G. M. Bokoch. 2005. GDIs: central regulatory molecules in Rho GTPase activation. Trends Cell Biol 15:356-63.

6. DerMardirossian, C., A. Schnelzer, and G. M. Bokoch. 2004. Phosphorylation of RhoGDI by Pakl mediates dissociation of Rac GTPase. Mol Cell 15:117-27.

7. Dovas, A., and J. R. Couchman. 2005. RhoGDI: multiple functions in the regulation of Rho family GTPase activities. Biochem J 390:1-9.

8. Dransart, E., B. Olofsson, and J. Cherfils. 2005. RhoGDIs revisited: novel roles in Rho regulation. Traffic 6:957-66.

9. Etienne-Manneville, S., and A. Hall. 2002. Rho GTPases in cell biology. Nature 420:629-35.

10. Faure, J., and M. C. Dagher. 2001. Interactions between Rho GTPases and Rho GDP dissociation inhibitor (Rho-GDI). Biochimie 83:409-14.

11. Golovanov, A. P., T. H. Chuang, C. DerMardirossian, I. Barsukov, D. Hawkins, R. Badii, G. M. Bokoch, L. Y. Lian, and G. C. Roberts. 2001. Structure-activity relationships in flexible protein domains: regulation of rho GTPases by RhoGDI and D4 GDI. J Mol Biol 305:121-35.

12. Gosser, Y. Q., T. K. Nomanbhoy, B. Aghazadeh, D. Manor, C. Combs, R. A. Cerione, and M. K. Rosen. 1997. C-terminal binding domain of Rho GDP-dissociation inhibitor directs N-terminal inhibitory peptide to GTPases. Nature 387:814-9.

13. Hirao, M., N. Sato, T. Kondo, S. Yonemura, M. Monden, T. Sasaki, Y. Takai, S. Tsukita, and S. Tsukita. 1996. Regulation mechanism of ERM (ezrin/radixin/moesin) protein/plasma membrane association: possible involvement of phosphatidylinositol turnover and Rho-dependent signaling pathway. J Cell Biol 135:37-51.

14. Hoffman, G. R., N. Nassar, and R. A. Cerione. 2000. Structure of the Rho family GTP-binding protein Cdc42 in complex with the multifunctional regulator RhoGDI. Cell 100:345-56.

15. Holinstat, M., N. Knezevic, M. Broman, A. M. Samarel, A. B. Malik, and D. Mehta. 2006. Suppression of RhoA activity by focal adhesion kinase-induced activation of p!90RhoGAP: role in regulation of endothelial permeability. J Biol Chem 281 :2296-305.

16. Holinstat, M., D. Mehta, T. Kozasa, R. D. Minshall, and A. B. Malik. 2003. Protein kinase Calpha-induced pi ISRhoGEF phosphorylation signals endothelial cytoskeletal rearrangement. J Biol Chem 278:28793-8.

17. Kotani, H., K. Takaishi, T. Sasaki, and Y. Takai. 1997. Rho regulates association of both the ERM family and vinculin with the plasma membrane in MDCK cells. Oncogene 14:1705-13.

18. Kouklis, P., M. Konstantoulaki, S. Vogel, M. Broman, and A. B. Malik 2004. Cdc42 regulates the restoration of endothelial barrier function. Circ Res 94:159-66.

19. Kozasa, T., X. Jiang, M. J. Hart, P. M. Sternweis, W. D. Singer, A. G. Oilman, G. Bollag, and P. C. Sternweis. 1998. pi 15 RhoGEF, a GTPase activating protein for Galpha12 and Galpha13. Science 280:2109-11.

20. Lian, L. Y., I. Barsukov, A. P. Golovanov, D. I. Hawkins, R. Badii, K. H. Sze, N. H. Keep, G. M. Bokoch, and G. C. Roberts. 2000. Mapping the binding site for the GTP-binding protein Rac-1 on its inhibitor RhoGDI-1. Structure 8:47-55.

21. Longenecker, K., P. Read, U. Derewenda, Z. Dauter, X. Liu, S. Garrard, L. Walker, A. V. Somlyo, R. K. Nakamoto, A. P. Somlyo, and Z. S. Derewenda. 1999. How RhoGDI binds Rho. Acta Crystallogr D Biol Crystallogr 55:1503-15.

22. Mehta, D., and A. B. Malik. 2006. Signaling mechanisms regulating endothelial permeability. Physiol Rev 86:279-367.

23. Mehta, D., A. Rahman, and A. B. Malik. 2001. Protein kinase C-alpha signals rho-guanine nucleotide dissociation inhibitor phosphorylation and rho activation and regulates the endothelial cell barrier function. J Biol Chem 276:22614-20.

24. Moers, A., N. Wettschureck, S. Gruner, B. Nieswandt, and S. Offermanns. 2004. Unresponsiveness of platelets lacking both Galpha(q) and Galpha(13). Implications for collagen-induced platelet activation. J Biol Chem 279:45354-9.

25. Nobes, C. D., and A. Hall. 1999. Rho GTPases control polarity, protrusion, and adhesion during cell movement. J Cell Biol 144: 1235-44.

26. Olofsson, B. 1999. Rho guanine dissociation inhibitors: pivotal molecules in cellular signalling. Cell Signal 11:545-54.

27. Ridley, A. J. 1999. Rho family proteins and regulation of the actin cytoskeleton. Prog Mol Subcell Biol 22:1-22.

28. Takahashi, K, T. Sasaki, A. Mammoto, K. Takaishi, T. Kameyama, S. Tsukita, and Y. Takai. 1997. Direct interaction of the Rho GDP dissociation inhibitor with ezrin/radixin/moesin initiates the activation of the Rho small G protein. J Biol Chem 272:23371-5.

29. Takai, Y., T. Sasaki, and T. Matozaki. 2001. Small GTP-binding proteins. Physiol Rev 81:153-208.

30. Takaishi, K., A. Kikuchi, S. Kuroda, K. Kotani, T. Sasaki, and Y. Takai. 1993. Involvement of rho p21 and its inhibitory GDP/GTP exchange protein (rho GDI) in cell motility. Mol Cell Biol 13:72-9.

31. Takaishi, K., T. Sasaki, T. Kameyama, S. Tsukita, S. Tsukita, and Y. Takai. 1995. Translocation of activated Rho from the cytoplasm to membrane ruffling area, cell-cell adhesion sites and cleavage furrows. Oncogene 11:39-48

32. Takaishi, K., T. Sasaki, M. Kato, W. Yamochi, S. Kuroda, T. Nakamura, M. Takeichi, and Y. Takai. 1994. Involvement of Rho p21 small GTP-binding protein and its regulator in the HGF-induced cell motility. Oncogene 9:273-9.

33. Takaishi, K., T. Sasaki, H. Kotani, H. Nishioka, and Y. Takai. 1997. Regulation of cell-cell adhesion by rac and rho small G proteins in MDCK cells. J Cell Biol 139:1047-59.

34. Vogt, S., R. Grosse, G. Schultz, and S. Offermanns. 2003. Receptor-dependent RhoA activation in G 12/G13-deficient cells: genetic evidence for an involvement of Gq/G1 1. J Biol Chem 278:28743-9.

35. Vouret-Craviari, V., C. Bourcier, E. Boulter, and E. van Obberghen-Schilling. 2002. Distinct signals via Rho GTPases and Src drive shape changes by thrombin and sphingosine-1-phosphate in endothelial cells. J Cell Sci 115:2475-84.

36. Wojciak-Stothard, B., and A. J. Ridley. 2002. Rho GTPases and the regulation of endothelial permeability. Vascul Phannacol 39:187-99.

Those skilled in the art will recognize, or be able to ascertain using no more than routine experimentation, many equivalents to the specific embodiments of the invention described herein. Such equivalents are intended to be encompassed by the following claims.

We claim:

1. A method for treating an inflammatory disease comprising delivering to a patient a compound or pharmaceutical composition in an amount effective to modulate GDI-1 activity.

2. The method of claim 1, wherein the compound inhibits phosphorylation of GDI-1.

3. The method of claim 1, wherein the compound increases phosphorylation of GDI-1.

4. The method of claim 3, wherein the increase in GDI-1 activity results in increased endothelial permeability.

5. The method of claim 1, wherein the inflammatory disease is selected from the group consisting of acute lung injury, ARDS, inflammatory bowel disease, arthritis, atherosclerosis, asthma, allergy, inflammatory kidney disease, circulatory shock, multiple sclerosis, chronic obstructive pulmonary disease, skin inflammation, periodontal disease, psoriasis and T cell-mediated diseases of immunity.

6. A screening method for identifying a compound which inhibits the phosphorylation of GDI-1 comprising (i) contacting a test compound, GDI-1 and a kinase; and (ii) determining phosphorylation of GDI-1 in the presence and the absence of the test compound; a decrease in phosphorylation in the presence relative to the absence of the test compound being indicative that the test compound inhibits the phosphorylation of GDI-1 by the kinase.

7. The method of claim 6, wherein the kinase is PKCα.

8. A screening method for identifying a compound which increases the phosphorylation of GDI-1 comprising (i) contacting a test compound, GDI-1 and a kinase; and (ii) determining phosphorylation of GDI-1 in the presence and the absence of the test compound; an increase in phosphorylation in the presence relative to the absence of the test compound being indicative that the test compound increases the phosphorylation of GDI-1 by the kinase.

9. The method of claim 8, wherein the kinase is PKCα.

10. The method of claim 2, wherein the inhibition of GDI-1 phosphorylation results in decreased RhoA activity.

11. The method of claim 6 or claim 8, wherein the GDI-1 of step (i) is bound to Rho-A.

12. The method of claim 3, wherein the increased phosphorylation of GDI-1 results in increased RhoA activity.

13. The method of claim 10, wherein the decreased RhoA activity results in decreased endothelial permeability.

14. The method of claim 12, wherein the increased RhoA activity results in increased endothelial permeability.
